(19) United States
(12) Patent Application Publication
(10) Pub. No.: US 2008/0139647 A1
(43) Pub. Date: Jun. 12, 2008

(54) COSMETIC PREPARATIONS

(76) Inventor: Gerald L. Hanson, Kingston, WA (US)

Correspondence Address: BARNES & THORNBURG LLP, 11 SOUTH MERIDIAN, INDIANAPOLIS, IN, 46204, US

(21) Appl. No.: 11/868,773

(22) Filed: Oct. 8, 2007

Related U.S. Application Data
(60) Provisional application No. 60/852,073, filed on Oct. 16, 2006.

(51) Int. Cl. A61K 8/37; A61Q 99/00

(52) U.S. Cl. 514/474

(57) ABSTRACT

A cosmetic preparation comprising about 50% to about 80% by weight of the finished preparation silicone fluid having a kinematic viscosity in the range of about 10,000 cs to about 15,000 cs, about 20% to about 40% by weight of the finished preparation silicone fluid having a kinematic viscosity in the range of about 25 cs to about 75 cs, about 20% to about 40% by weight of the finished preparation silicone fluid having a kinematic viscosity in the range of about 1 cs to about 10 cs, about 0.25% to about 10% by weight of the finished preparation vinyl silicone crosspolymer, and about 0.25% to about 10% by weight of the finished preparation an ascorbic acid ester.

### CROSS-REFERENCE TO RELATED APPLICATIONS

This application claims the benefit under 35 U.S.C. § 119(e) of the Oct. 16, 2006 filing date of U.S. Ser. No. 60/852,073. The disclosure of U.S. Ser. No. 60/852,073 is hereby incorporated herein by reference.

### FIELD OF THE INVENTION

This invention relates generally to cosmetic preparations. It is disclosed in the context of a cosmetic preparation for application to, for example, hypertrophic scars, keloid scars and erythema. However, it is believed to be useful in other applications as well.

### BACKGROUND OF THE INVENTION

Cosmetic preparations are known. There are, for example, the cosmetic preparations described in U.S. Pat. No. 5,741,509 and the references cited therein. This listing is not intended to be a representation that a complete search of all relevant art has been made, or that no more pertinent art than that listed exists, or that the listed art is material to patentability. Nor should any such representation be inferred.

### DISCLOSURE OF THE INVENTION

According to an aspect of the invention, a cosmetic preparation comprises about 1% to about 80% by weight of the finished preparation silicone fluid having a kinematic viscosity in the range of about 10,000 centistokes (cs) to about 15,000 cs, about 1% to about 80% by weight of the finished preparation silicone fluid having a kinematic viscosity in the range of about 25 cs to about 75 cs, about 1% to about 80% by weight of the finished preparation silicone fluid having a kinematic viscosity in the range of about 1 cs to about 10 cs, about 1% to about 80% by weight of the finished preparation vinyl silicone crosspolymer, and about 0.25% to about 80% by weight of the finished preparation an ascorbic acid ester.

Illustratively according to this aspect of the invention, the cosmetic preparation comprises about 1% to about 80% by weight of the finished preparation tetrahexyldecyl ascorbate.

Further illustratively according to this aspect of the invention, the cosmetic preparation comprises about 0.5% to about 10% by weight of the finished preparation tetrahexyldecyl ascorbate.

Illustratively according to this aspect of the invention, the cosmetic preparation comprises about 1% to about 80% by weight of the finished preparation silicone fluid having a kinematic viscosity about 12,500 cs.

Further illustratively according to this aspect of the invention, the cosmetic preparation comprises about 50% to about 80% by weight of the finished preparation silicone fluid having a kinematic viscosity about 12,500 cs.

Illustratively according to this aspect of the invention, the cosmetic preparation comprises about 1% to about 80% by weight of the finished preparation silicone fluid having a kinematic viscosity about 50 cs.

Further illustratively according to this aspect of the invention, the cosmetic preparation comprises about 20% to about 40% by weight of the finished preparation silicone fluid having a kinematic viscosity about 50 cs.

Illustratively according to this aspect of the invention, the cosmetic preparation comprises about 1% to about 80% by weight of the finished preparation silicone fluid having a kinematic viscosity about 5 cs.

Further illustratively according to this aspect of the invention, the cosmetic preparation comprises about 20% to about 40% by weight of the finished preparation silicone fluid having a kinematic viscosity about 5 cs.

### DETAILED DESCRIPTIONS OF ILLUSTRATIVE EMBODIMENTS

The invention may best be understood by referring to the following detailed description and examples which illustrate the invention.

A cosmetic preparation is prepared by combining about 1% to about 80% by weight of the finished preparation silicone fluid having a kinematic viscosity in the range of about 10,000 cs to about 15,000 cs, about 1% to about 80% by weight of the finished preparation silicone fluid having a kinematic viscosity in the range of about 25 cs to about 75 cs, about 1% to about 80% by weight of the finished preparation silicone fluid having a kinematic viscosity in the range of about 1 cs to about 10 cs, about 1% to about 80% by weight of the finished preparation vinyl silicone crosspolymer, and about 1% to about 80% by weight of the finished preparation an ascorbic acid ester.

Further illustratively, the cosmetic preparation comprises about 1% to about 80% by weight of the finished preparation vinyl silicone crosspolymer.

Illustratively, the silicones are in the form of dimethicones, that is, silicone oils comprising dimethylsiloxane polymers, usually incorporated into petrolatum bases or other nongreasy preparations. Such silicones are generally used for the protection of normal skin against, for example, industrial, skin irritants. Illustratively, the vinyl silicone crosspolymer is in the form of vinyl dimethicone crosspolymer.

Illustratively, the about 1% to about 80% by weight of the finished preparation silicone fluid having a kinematic viscosity in the range of about 10,000 cs to about 15,000 cs comprises silicone fluid having a kinematic viscosity of about 12,500 cs. Illustratively, the about 1% to about 80% by weight of the finished preparation silicone fluid having a kinematic viscosity in the range of about 10,000 cs to about 15,000 cs comprises Dow Corning® 200® fluid, kinematic viscosity about 12,500 cs, available from Dow Corning Corporation, Midland, Mich.

Illustratively, the about 1% to about 80% by weight of the finished preparation silicone fluid having a kinematic viscosity in the range of about 25 cs to about 75 cs comprises silicone fluid having a kinematic viscosity of about 50 cs. Illustratively, the about 1% to about 80% by weight of the finished preparation silicone fluid having a kinematic viscosity in the range of about 25 cs to about 75 cs comprises Dow Corning® 200® fluid, kinematic viscosity about 50 cs, available from Dow Corning Corporation, Midland, Mich.

Illustratively, the about 1% to about 80% by weight of the finished preparation silicone fluid having a kinematic viscosity in the range of about 1 cs to about 10 cs comprises silicone fluid having a kinematic viscosity of about 5 cs. Illustratively, the about 1% to about 80% by weight of the finished preparation silicone fluid having a kinematic viscosity in the range of about 1 cs to about 10 cs comprises Dow Corning® 200® fluid, kinematic viscosity about 5 cs, available from Dow Corning Corporation, Midland, Mich.

Illustratively, the about 1% to about 80% by weight of the finished preparation vinyl silicone crosspolymer comprises about 1% to about 80% by weight of the finished preparation Dow Corning® 2502® fluid, available from Dow Corning Corporation, Midland, Mich.

Illustratively, the about 1% to about 80% by weight of the finished preparation ascorbate ester comprises about 1% to about 80% by weight of the finished preparation tetrahexyldecyl ascorbate.

### Example 1

In a specific, but only illustrative, example, about 70% by weight of the finished preparation about 12,500 cs silicone fluid, about 14% by weight of the finished preparation about 50 cs silicone fluid, about 14% by weight of the finished preparation about 5 cs silicone fluid, about 1% by weight of the finished preparation vinyl silicone crosspolymer, and about 1% by weight of the finished preparation tetrahexyldecyl ascorbate are blended to create a cosmetic preparation for application to, and for use in treatment of, for example, hypertrophic scars, keloid scars and erythema.

### Example 2

In another example, about 68.5% by weight of the finished preparation about 12,500 cs silicone fluid, about 15% by weight of the finished preparation about 50 cs silicone fluid, about 14.5% by weight of the finished preparation about 5 cs silicone fluid, about 1% by weight of the finished preparation silicone/vinyl silicone crosspolymer, about 0.5% by weight of the finished preparation tetrahexyldecyl ascorbate and about 0.5% by weight of the finished preparation orange oil are blended to create a cosmetic preparation for application to, and for use in treatment of, for example, hypertrophic scars, keloid scars and erythema. An illustrative orange oil is Bell Flavors & Fragrances 10-fold natural orange oil.

What is claimed is:

1. A cosmetic preparation comprising about 1% to about 80% by weight of the finished preparation silicone fluid having a kinematic viscosity in the range of about 10,000 cs to about 15,000 cs, about 1% to about 80% by weight of the finished preparation silicone fluid having a kinematic viscosity in the range of about 25 cs to about 75 cs, about 1% to about 80% by weight of the finished preparation silicone fluid having a kinematic viscosity in the range of about 1 cs to about 10 cs, and about 0.25% to about 80% by weight of the finished preparation an ascorbic acid ester.

2. The cosmetic preparation of claim 1 wherein the ascorbic acid ester comprises tetrahexyldecyl ascorbate.

3. The cosmetic preparation of claim 2 wherein the silicone fluid having a kinematic viscosity in the range of about 10,000 cs to about 15,000 cs comprises silicone fluid having a kinematic viscosity of about 12,500 cs.

4. The cosmetic preparation of claim 2 wherein the silicone fluid having a kinematic viscosity in the range of about 25 cs to about 75 cs comprises silicone fluid having a kinematic viscosity of about 50 cs.

5. The cosmetic preparation of claim 2 wherein the silicone fluid having a kinematic viscosity in the range of about 1 cs to about 10 cs comprises silicone fluid having a kinematic viscosity of about 5 cs.

6. The cosmetic preparation of claim 2 wherein the about 0.25% to about 80% by weight of the finished preparation an ascorbic acid ester comprises about 0.25% to about 10% by weight of the finished preparation tetrahexyldecyl ascorbate.

7. The cosmetic preparation of claim 6 wherein the silicone fluid having a kinematic viscosity in the range of about 10,000 cs to about 15,000 cs comprises silicone fluid having a kinematic viscosity of about 12,500 cs.

8. The cosmetic preparation of claim 6 wherein the silicone fluid having a kinematic viscosity in the range of about 25 cs to about 75 cs comprises silicone fluid having a kinematic viscosity of about 50 cs.

9. The cosmetic preparation of claim 6 wherein the silicone fluid having a kinematic viscosity in the range of about 1 cs to about 10 cs comprises silicone fluid having a kinematic viscosity of about 5 cs.

10. The cosmetic preparation of claim 1 comprising about 0.25% to about 10% by weight of the finished preparation an ascorbic acid ester.

11. The cosmetic preparation of claim 10 wherein the silicone fluid having a kinematic viscosity in the range of about 10,000 cs to about 15,000 cs comprises silicone fluid having a kinematic viscosity of about 12,500 cs.

12. The cosmetic preparation of claim 10 wherein the silicone fluid having a kinematic viscosity in the range of about 25 cs to about 75 cs comprises silicone fluid having a kinematic viscosity of about 50 cs.

13. The cosmetic preparation of claim 10 wherein the silicone fluid having a kinematic viscosity in the range of about 1 cs to about 10 cs comprises silicone fluid having a kinematic viscosity of about 5 cs.

14. The cosmetic preparation of claim 1 wherein the silicone fluid having a kinematic viscosity in the range of about 10,000 cs to about 15,000 cs comprises silicone fluid having a kinematic viscosity of about 12,500 cs.

15. The cosmetic preparation of claim 1 wherein the silicone fluid having a kinematic viscosity in the range of about 25 cs to about 75 cs comprises silicone fluid having a kinematic viscosity of about 50 cs.

16. The cosmetic preparation of claim 1 wherein the silicone fluid having a kinematic viscosity in the range of about 1 cs to about 10 cs comprises silicone fluid having a kinematic viscosity of about 5 cs.

17. The cosmetic preparation of claim 1 further comprising about 0.25% to about 80% by weight of the finished preparation vinyl silicone crosspolymer.

18. The cosmetic preparation of claim 17 wherein the silicone fluid having a kinematic viscosity in the range of about 10,000 cs to about 15,000 cs comprises silicone fluid having a kinematic viscosity of about 12,500 cs.

19. The cosmetic preparation of claim 17 wherein the silicone fluid having a kinematic viscosity in the range of about 25 cs to about 75 cs comprises silicone fluid having a kinematic viscosity of about 50 cs.

20. The cosmetic preparation of claim 17 wherein the silicone fluid having a kinematic viscosity in the range of about 1 cs to about 10 cs comprises silicone fluid having a kinematic viscosity of about 5 cs.

21. The cosmetic preparation of claim 17 comprising about 0.25% to about 10% by weight of the finished preparation an ascorbic acid ester.

22. The cosmetic preparation of claim 17 comprising about 0.25% to about 10% by weight of the finished preparation a fragrance-providing composition.

23. The cosmetic preparation of claim 22 comprising about 0.25% to about 5% by weight of the finished preparation orange oil.

24. A cosmetic preparation comprising about 50% to about 80% by weight of the finished preparation silicone fluid having a kinematic viscosity in the range of about 10,000 cs to about 15,000 cs, about 20% to about 40% by weight of the finished preparation silicone fluid having a kinematic viscosity in the range of about 25 cs to about 75 cs, about 20% to about 40% by weight of the finished preparation silicone fluid having a kinematic viscosity in the range of about 1 cs to about 10 cs, about 0.25% to about 10% by weight of the finished preparation vinyl silicone crosspolymer, and about 0.25% to about 10% by weight of the finished preparation an ascorbic acid ester.

25. The cosmetic preparation of claim 24 comprising about 0.25% to about 10% by weight of the finished preparation a fragrance-providing composition.

26. The cosmetic preparation of claim 25 comprising about 0.25% to about 5% by weight of the finished preparation orange oil.
